(19) United States
(12) Patent Application Publication
(10) Pub. No.: US 2014/0030314 A1
(43) Pub. Date: Jan. 30, 2014

(54) TREATMENT OF SKIN DISEASE

(71) Applicants: Brian G. Larson, Alpine, UT (US); David Larson, Pleasant Grove, UT (US)

(72) Inventors: Brian G. Larson, Alpine, UT (US); David Larson, Pleasant Grove, UT (US)

(21) Appl. No.: 13/740,879

(22) Filed: Jan. 14, 2013

Related U.S. Application Data
(60) Provisional application No. 61/586,485, filed on Jan. 13, 2012.

(51) Int. Cl. A61K 33/40; A61K 45/06; A61K 33/38

(52) U.S. Cl. CPC A61K 33/40; A61K 33/38; A61K 45/06
USPC 424/443; 424/614; 424/616; 424/85.4; 424/489

(57) ABSTRACT

The present invention is drawn to compositions, systems, and methods of treating skin disease. In one example, the composition includes a peroxygen, a transition metal or alloy thereof, and optionally, an alcohol and/or a drug. The composition can be packaged as part of a two-part system.

### BACKGROUND OF THE INVENTION

Acne and other skin disease and infection afflict many people, both on the face and elsewhere on the body. For example, acne is a skin condition that causes whiteheads, blackheads, and inflamed red growths in the form of papules, pustules, and cysts. Acne can occur when pores on the surface of the skin become blocked or clogged. The pores are present as an opening to a follicle, which contains a hair and an oil gland which act to lubricate the skin and help remove old skin cells. Thus, the oil that is present, along with dirt, debris, bacteria, cells, etc., often work together to cause the blockage. When the blockage breaks open, the material inside causes swelling and red bumps to form, which often lead to firm and painful cysts.

There are also many other type of infections that have a negative impact on skin health, including inflammation, viral, bacterial, and fungal infections. Continued improvement in the treatment of skin afflicted with these and other types of diseases would be a benefit to those suffering from these types of conditions. Many drugs, including topically and orally administered drugs, have been used to treat skin infections and diseases to some success. However, it would be an advancement in the art to provide alternative treatments that may or may not include a drug which would enhance skin health and treatment generally.

### SUMMARY OF THE INVENTION

In accordance with this, the present disclosure is drawn to compositions, systems, and methods of treating skin disease. In one embodiment, a composition suitable for treating skin disease can comprise water, from 0.0001 wt % to 10.0 wt % of a peroxygen; from 0.0001 ppm to 50,000 ppm by weight of a transition metal or alloy thereof; and a drug suitable for treating the skin disease. In one example, the composition is in an active state where at least two components of the composition are not in equilibrium at the time of skin application. A related method includes treating a skin disease comprising applying the composition directly to a skin site afflicted with the skin disease.

In another embodiment, a system suitable for treating skin disease can comprise a first container containing Part A of a two-part solution, and a second container containing Part B of the two-part solution. Part A can include a transition metal or alloy thereof and Part B can include water and a peroxygen (or vice versa, as the letter “A” or “B” is used merely for convenience). A drug suitable for treating the skin disease can be present in at least one of Part A, Part B, or a third formulation. Upon combining Part A and Part B, a reacting formulation is formed that, in combination with the drug, is effective for treating the skin disease. In a related method, treating the skin disease can include obtaining the system, combining Part A and Part B in the presence of the drug to form a reacting formulation, and applying the reacting formulation to a skin site afflicted with the skin disease.

In another embodiment, a system suitable for treating skin disease can comprise a first container containing Part A of a two-part solution and a second container containing Part B of the two-part solution. Part A can include a transition metal or alloy thereof and Part B can include water and a peroxygen (or vice versa, as the letter “A” or “B” is used merely for convenience). In this embodiment, one of Part A and Part B can be soaked into a skin wipe, and the other of Part A and Part B can present in a dispenser adapted to dispense its solution onto the skin wipe to form a reacting formulation that is effective for treating the skin disease. More specifically, the dispenser can be adapted to dispense its solution onto less than all of the plurality of skin wipes (one wipe or just a few wipes) so that not all of the plurality of skin wipes are activated.

In another embodiment, a method of treating a skin disease can comprise obtaining a two-part solution comprising Part A which includes a transition metal or alloy thereof, and Part B which includes water and a peroxygen (or vice versa, as the letter “A” or “B” is used merely for convenience). Additional steps include combining Part A with Part B to form a reacting formulation and applying the reacting formulation to the skin disease. The two-part solution can include a drug provided in Part A, Part B, or by a third formulation containing the drug.

Additional features and advantages of the invention will be apparent from the detailed description that follows, which illustrates, by way of example, features of the invention.

### DETAILED DESCRIPTION

Reference will now be made to the exemplary embodiments, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention. It is also to be understood that the terminology used herein is used for the purpose of describing particular embodiments only. The terms are not intended to be limiting unless specified as such.

It must be noted that, as used in this specification and the appended claims, the singular forms “a,” “an,” and “the” include plural referents unless the content clearly dictates otherwise.

The terms “solution,” “composition” and “formulation” are also used throughout the specification to describe the compositions of the present disclosure. However, as these “solutions” can include colloidal transition metals, these compositions can also be described as dispersions or suspensions. As the continuous phase is typically a solution, and the transition metal can be present in ionic and/or colloidal form (and typically in small amounts and sizes), for convenience, these compositions will typically be referred to as “solutions,” “compositions” or “formulations” interchangeably. Further, sometimes a solution is referred to as a “resultant” solution or composition. This is to provide added clarity that the solution is a product of the mixing of a two-part system. This being stated, the terms “solution” and “resultant solution” can be used interchangeably herein as is typically clear from the context of the discussion.

The term “reacting formulation” refers to compositions that are not at equilibrium, and in fact, often are actively reacting. For example, upon admixing the two-part formulation of the present disclosure, components form a reacting admixture that takes some time to come to equilibrium. During this reactive state after bringing the two-parts together, the resultant reacting formulation is more highly active for treating skin disease in accordance with embodiments of the present disclosure. Once equilibrium is reached, the formulation is not as effective at treating skin disease as it was while actively reacting.

The term “peroxygen” refers to any compound containing a dioxygen (O—O) bond. Dioxygen bonds, particularly bivalent O—O bonds, are readily cleavable, thereby allowing compounds containing them to act as powerful oxidizers. Non-limiting examples of classes of peroxygen compounds include peracids, peracid salts, and peroxides, such as hydrogen peroxide or metal peroxides.

When referring to the term “alloy,” it is understood that individual colloidal or metallic particles can be in the form of composites of multiple metals, or alloys can also include co-dispersions of multiple metals as separate particles.

The term “two-part” when referring to the systems of the present disclosure is not limited to systems having only two parts. For example, the system can be a concentrate, and thus, is actually a three-part system, e.g., a first part including transition metal and optionally an alcohol and/or drug, a second part including a peroxygen and optionally an alcohol and/or drug, and a third part of a diluting solvent for diluting the first part, the second part, and/or the resultant solution. The third part could also include a drug, for example. Non-limiting examples of diluting solvents include water, alcohols, or combinations thereof. When the diluting solvent is an alcohol, it can, but need not be the same alcohol or mixture of alcohols which are present in the first “part” of the system. Thus, “two-part’ is specifically defined herein to mean, at least two-parts, unless the context dictates otherwise. Also, when referring to “Part A” or “Part B,” it is noted that the letter “A” or “B” is used merely for convenience, and does not infer which other co-ingredients may be present in a specific Part A or Part B formulation. Thus, “A” and “B” shall be interpreted to have no specific inference other than to identify an ingredient is from “this” part or the “other” part.

The term “container” refers to traditional containers such as tubes, dispensers, bottles, sprayers, etc. However, this term is to be viewed to be viewed more broadly to include fabrics (wipes), bandages, wrappings (foil, paper, etc.). Thus, anything capable of “containing” a fluid in accordance with embodiments of the present disclosure can be considered a container.

The term “drug” refers to any bioactive agent or agents which can be used to effectively treat skin disease or other skin infection or affliction, e.g., inflammatory, viral, fungal, bacterial, etc. Often, a single drug or active agent can be effective in treating multiple disease or infection types, or combination of multiple drugs can be used to treat a single or multiple infection types. Thus, no particular drug or combination thereof is to be associated specifically with a specific skin disease.

The term “subject” refers to any animal. In particular, subjects can be mammals, and more particularly humans.

The term “skin” includes human skin, nail, and mucosal surfaces that can suffer from various forms of disease and infection and are usually at least partially exposed to the environment, such as skin, nails, lips, and mucosa.

The term “skin disease” refers to any of a number of skin ailments and infections that afflict the skin surface or deeper skin tissue, including inflammatory skin disease (e.g., acne, eczema, dermatitis, poison ivy, psoriasis, pyoderma gangrenosum, rosacea, hives, inflamed burns, etc.); bacterial skin infection (e.g., impetigo, folliculitis, furunculosis, carbunculosis, eethyma, erysipelas, cellulitis, necrotizing fasciitis, etc.); fungal and yeast infection (e.g., dermatophytosis, candidiasis, tinea, athlete's foot, nail fungal infection, diaper rash, etc.); viral infection (e.g., herpes simplex, herpes zoster, cold sores, warts, molluscum contagiosum, etc.); and infection caused by small macro organisms such as mites (e.g., face mites such as demodex folliculorum, Demodex brevis, Demodex canis, etc.), insects, animals (bites), etc.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight ratio range of about 1 wt % to about 20 wt % should be interpreted to include not only the explicitly recited limits of 1 wt % and about 20 wt %, but also to include individual weights such as 2 wt %, 11 wt %, 14 wt %, and sub-ranges such as 10 wt % to 20 wt %, 5 wt % to 15 wt %, etc.

It is noted that when a range or value is given with respect to weight percent (wt %), the weight percent that is referred to is that in the resultant composition or formulation after the two-part system is brought together unless clearly stated otherwise. Thus, if it is stated that a drug is present in a formulation at from 3 wt % to 8 wt %, that indicates that the final composition that is applied the skin has drug present within that weight ratio range. This is primarily applicable to the two-part embodiments described herein, as one-part systems would not create any confusion as to the applicable weight ratio range. Thus, it is understood that the initial drug or other ingredient present in one of the two-parts may be greater than that in the resultant formulation, and may be outside of the range described in the composition that will ultimately be applied to the skin. Alternatively, in some instances, a weight percentage will be given and clearly labeled as being a weight percentage of one specific part of a two-part system (Part A or Part B), e.g., see certain examples. In those instances, the weight percentages shall be as indicated. Thus, in these “two-part” embodiments, it is notable that the concentrations of each ingredient can be described in the context of concentration in the first or second composition (when specifically indicated), or the resultant solution or composition (as a default). The concentration of a compound in the first or second liquid composition will usually be lower in the resultant composition or solution than in the first or second liquid composition, as the amount typically gets diluted by the other part of the system. That being stated, this is not always the case, depending on the ingredients in the other portion of the two-part system. For example, if an ingredient is generated by a reaction, the amount may actually increase when the two-part system is combined to form the resultant composition, e.g., peracid and peroxide chemistry.

With this in mind, the present disclosure provides compositions, systems, and methods for treating and/or preventing skin disease of various types. In one embodiment, a composition suitable for treating skin disease can comprise water, from 0.0001 wt % to 10.0 wt % of a peroxygen; from 0.0001 ppm to 50,000 ppm by weight of a transition metal or alloy thereof; and a drug suitable for treating the skin disease. In one example, the composition is in an active state where at least two components of the composition are not in equilibrium at the time of use. A related method can include treating a skin disease comprising applying the composition directly to a skin site afflicted with the skin disease.

In one specific example, the compositions or formulations of the present disclosure can be prepared to include from 0.1 wt % to 10 wt % salicylic acid as the drug, from 0.1 wt % to 10 wt % hydrogen peroxide as the peroxygen, from 1 ppm to 20,000 ppm by weight colloidal silver as a portion of the transition metal, and from 1 ppm to 20,000 ppm by weight colloidal zinc as a second portion of the transition metal. Optional ingredients in addition can include from 0.5 wt % to 25 wt % glycerol and from 0.5 wt % to 15 wt % coconut oil.

In another embodiment, a system suitable for treating skin disease can comprise a first container containing Part A of a two-part solution, and a second container containing Part B of the two-part solution. Part A can include a transition metal or alloy thereof and Part B can include water and a peroxygen. A drug suitable for treating the skin disease can be present in at least one of Part A, Part B, or a third formulation. Upon combining Part A and Part B, a reacting formulation is formed that, in combination with the drug, is effective for treating the skin disease. In a related method, treating the skin disease can include obtaining the system, combining Part A and Part B in the presence of the drug to form a reacting formulation, and applying the reacting formulation to a skin site afflicted with the skin disease.

In another embodiment, a system suitable for treating skin disease can comprise a first container containing Part A of a two-part solution and a second container containing Part B of the two-part solution. Part A can include a transition metal or alloy thereof and Part B can include water and a peroxygen. In this embodiment, one of Part A and Part B can be soaked into a skin wipe, and the other of Part A and Part B can present in a dispenser adapted to dispense its solution onto the skin wipe to form a reacting formulation that is effective for treating the skin disease. More specifically, the dispenser adapted can be configured to dispense its solution onto less than all of the plurality of skin wipes (one wipe or just a few wipes) so that not all of the plurality of skin wipes are activated.

In another embodiment, a method of treating a skin disease can comprise obtaining a two-part solution comprising Part A which includes a transition metal or alloy thereof, and Part B which includes water and a peroxygen. Additional steps include combining Part A with Part B to form a reacting formulation and applying the reacting formulation to the skin disease. The two-part solution can include a drug provided in Part A, Part B, or by a third formulation containing the drug.

In each of the various embodiments herein, whether discussing the compositions, systems, or methods, there may be some common features of each of these embodiments that further characterize options in accordance with principles discussed herein. Thus, discussions of the compositions, systems, or methods alone are also applicable to the other embodiments not specifically mentioned.

Though not required, typically, the therapeutic compositions of the present disclosure can be stored as a two-part system, and brought together prior to use, such as immediately prior to use, e.g., within 60 seconds, within 5 minutes of admixture, within 1 hour of admixture, within 24 hours of admixture, within 1 week of admixture. Longer periods of time may lead to the active ingredients becoming less effective, though to the extent that they remain effective for treating skin disease, any amount of time may be appropriate as long as the ingredients are effective for their intended use of treating skin disease. For example, a two-part system can be formulated to split up the ingredients between the two (or more) ingredients in any manner that preserves the activity of the ingredients for use when brought together. In accordance with this, the silver and the peroxygen are typically kept in separate containers to prevent premature activation of these ingredients prior to use.

As mentioned previously, the compositions of the present disclosure can comprise an aqueous vehicle including water, from 0.0001 wt % to 10.0 wt % of a peroxygen, from 0.0001 ppm to 50,000 ppm by weight of a transition metal or alloy thereof, and optionally, an alcohol and/or a drug. It is noted that the lower end of the range of the peroxygen in the administered aqueous composition can be modified to 0.05 wt % or 0.1 wt %, and/or the upper end of the range can be modified to 5 wt %, 3 wt %, or 1.5 wt % in accordance with specific embodiments of the present disclosure. Further, the concentration of the metal content, including ionic and/or colloidal content, can be modified to 10 ppm, 1 ppm, 0.1 ppm, 0.01, or 0.001 by weight at the lower end of the range, and/or to 10,000 ppm, 5,000 ppm, or 1,500 ppm by weight at the upper end of the range. It is also noted that the alcohol, can be present at from 0.0001 wt % to 95 wt %. This being stated, the lower end of the range of the alcohol can be modified to 0.05 wt % or 0.1 wt %, and the upper end of the range can be modified to 40 wt %, 30 wt %, 20 wt % or 10 wt % in accordance with specific embodiments of the present disclosure. Furthermore, the drug can be present ranging from 0.1 wt % to 20 wt %, without limitation. The lower end of the range of the drug can be modified to 0.5 wt % or 1 wt %, and the upper end of the range can be modified to 10 wt %, 5 wt %, or 2 wt % in accordance with specific embodiments of the present disclosure. As these ranges are merely exemplary, one skilled in the art could modify these ranges for a particular application, considering such things as the type of alcohol (polyhydric, mixtures, etc.); the type of peroxygen (peroxide, peracid, combination of peroxide/peracid, etc.); the type of metal (ionic, colloidal, alloy, etc.), the type of drug, and the particular skin disease being treated. Further, it is noted that any combination of these upper and lower limits for each of the ingredients are expressly included herein.

Though specific ingredients are described herein in detail, it is noted that there will also typically be an aqueous vehicle that includes water and optionally other ingredients, such as organic co-solvents, surfactants, and the like, so long as the additional ingredients are compatible with the intended compositions, systems, and methods of treatment.

In one specific example, the composition of the present disclosure can be prepared by admixing at least two-parts together in accordance with a preliminary step of admixing a first liquid composition and a second liquid composition to form the composition suitable for treating skin disease. The first liquid composition (or Part A) can comprise the transition metal or alloy thereof and optionally an alcohol, and the second liquid composition (or Part B) can comprise the peroxygen and optionally the drug. Alternatively, the first liquid composition (Part A) can comprise the peroxygen and optionally the alcohol and optionally the drug, and the second liquid composition (Part B) can comprise the transition metal or alloy thereof. Thus, it is not significant what ingredients are in Part A and what ingredients are in Part B, provided the parts that are reactive or interactive with one another are kept separate, e.g., colloidal metal separated from the peroxygen in some formulations, or the drug separated from any ingredient that would adversely impact its effectiveness. In some instances, it may be beneficial that the two-part system actually include three-parts if there are three ingredients that should be kept separate until just prior to use.

When a two-part solution is brought together, reactions occur that can also reduce or increase relative concentrations of given ingredients, e.g., in the case of peracid/peroxide compositions, the peroxide component of the peracid is rapidly converted into water and oxygen within minutes of activation, and ceases to exist in some cases. Additionally, such two-part embodiments can sometimes provide effective activation for a period of weeks, e.g., up to 60 days after activation or more, depending on the specific composition. Furthermore, whether two-part system or a single solution, these compositions can be prepared so that they are non-corrosive or non-toxic, and emit no emissions into the environment. Furthermore, these solutions can be prepared so that they pose no health or safety issues, since all of the ingredients (except for the drug in some instances) are essentially food grade after activation. For example, in the case of some two-part systems of peracids and peroxides, e.g., peroxyacetic acid and hydrogen peroxide, after activation by bringing the two-parts together, the dramatically altered chemical form of the peracid post-activation is no longer corrosive to the skin, exhibits no oral or inhalation toxicities, no dermal toxicities, and only mild irritation when sprayed directly into the eyes (no permanent damage to the eyes). If the desire is to treat the eye with the formulations of the present disclosure, safe formulations for eye application can also be prepared.

Turning to the compositional components more specifically, in embodiments where a drug used, examples of such drugs that are usable for one or a variety of skin diseases or conditions in accordance with the present disclosure include, without limitation, benzoyl peroxide, salicylic acid, sulfur, resorcinol, resorcinol monoacetate, amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, undecylenate, acyclovir, penciclovir, famciclovir, valacyclovir, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, rimantadine, zanamivir, erythromycin, clindamycin, tetracycline, bacitracin, neomycin, mupirocin, polymyxin B, quinolones, imiquimod, or combinations thereof. Other drugs that have a therapeutic effect with respect to certain types of skin disease can also be used and determined by one skilled in the art after considering the present disclosure with routine experimentation. Considerations for use would include activity with respect to a desired treatment of a skin disease, and compatibility with the other ingredients in the composition or during storage of at least one part of a two-part system.

In certain examples, there are certain specific drugs that can be used very effectively, with low side effects, on a variety of skin conditions, including but not limited to acne, warts, cold sores, eczema, psoriasis, athlete's foot, diaper rash, burns, etc., with acceptable results. These active agents or drugs include, without limitation, benzoyl peroxide (e.g., 2.5 wt % to 10 wt %), salicylic acid (e.g., 0.5 wt % to 2 wt %), sulfur (e.g., 3 wt % to 10 wt %), resorcinol (e.g., 1 wt % to 3 wt %), and resorcinol monoacetate (e.g., 2 wt % to 4 wt %). Furthermore, a combination of these active ingredients can also be used, such as about 2 wt % resorcinol combined with 3 wt % to 8 wt % sulfur, or about 3 wt % resorcinol monoacetate combined with 3 wt % to 8 wt % sulfur. Other combinations can also be effective. It is noted that these weight percentages given are provided for general guidance only, and can be expanded in accordance with examples of the present disclosure.

If an alcohol is present in the composition, or in one or both of Part A and Part B of the two-part system, in one example, the alcohol can be present at from about 0.0001 wt % to 95 wt %, with the upper end and lower end of the range being modifiable as described previously. Examples of alcohols that can be used include, but are limited to, aliphatic alcohols and other carbon-containing alcohols, having from 1 to 24 carbons (C₁-C₂₄ alcohol). It is to be noted that “C₁-C₂₄ alcohol” does not necessarily imply only straight chain saturated aliphatic alcohols, as other carbon-containing alcohols can also be used within this definition, including branched aliphatic alcohols, alicyclic alcohols, aromatic alcohols, unsaturated alcohols, as well as substituted aliphatic, alicyclic, aromatic, and unsaturated alcohols, etc. In one embodiment, the aliphatic alcohols can be C₁ to C₅ alcohols including methanol, ethanol, propanol and isopropanol, butanols, and pentanols, due to their availability and lower boiling points. This being stated, polyhydric alcohols can also be used effectively in enhancing the potency of the solution of the present disclosure, as well as provide some degree of added stabilization. Examples of polyhydric alcohols which can be used in the present disclosure include but are not limited to ethylene glycol (ethane-1,2-diol), glycerin (or glycerol, propane-1,2,3-triol), sorbitol, and propane-1,2-diol. Other non-aliphatic alcohols may also be used including but not limited to phenols and substituted phenols, erucyl alcohol, ricinolyl alcohol, arachidyl alcohol, capryl alcohol, capric alcohol, yl alcohol, lauryl alcohol (1-dodecanol), myristyl alcohol (1-tetradecanol), cetyl (or palmityl) alcohol (1-hexadecanol), stearyl alcohol (1-octadecanol), isostearyl alcohol, oleyl alcohol (cis-9-octadecen-1-ol), palmitoleyl alcohol, linoleyl alcohol (9Z,12Z-octadecadien-1-ol), elaidyl alcohol (9E-octadecen-1-ol), elaidolinoleyl alcohol (9E,12E-octadecadien-1-ol), linolenyl alcohol (9Z,12Z,15Z-octadecatrien-1-ol), elaidolinolenyl alcohol (9E,12E,15-E-octadecatrien-1-ol), combinations thereof, and the like.

In some embodiments, for practical considerations, methanol, ethanol, propanols, butanols, pentanols, and denatured alcohols (mixtures of ethanol and smaller amounts of methanol and other possible minor amounts of organics) can often be used because of their availability and cost. Glycerol or sorbitol can also be used in some embodiments. Since the desire is typically to provide a highly skin safe composition, then alcohols can be selected that satisfy this desire. When considering the amount of alcohol to use, one skilled in the art can stay within the above-described ranges, or modify these ranges for a particular application, considering such things as whether alcohol selected for use is polyhydric, whether the alcohol is food grade, mixtures of alcohols, etc.

Regarding the transition metal or alloy, a concentration in the range of 0.0001 ppm to 50,000 ppm by weight can be used and/or modified as described previously. The metal can be in ionic form (e.g. disassociate metal salt, metal ions from elemental metal, etc.) and/or in colloidal form. In one specific embodiment, the transition metal can be in a sub-micron form (i.e. dispersion of less than 1 μm metal colloidal particles). However, larger colloidal transition metal particles can also be used in certain applications. Typical transition metals that are desirable for use include Group VI to Group XI transition metals, and more preferably, can include Group X to Group XI transition metals. Alloys including at least one metal from the Group VI to Group XI metals can also be used. It is recognized that any of these metals will typically be oxidized to the corresponding cation in the presence of a peroxygen. However, with colloidal metals, typically, the surface is usually more susceptible to such oxidation. Further, when colloidal metals are dispersed in a colloidal solution, there is often an amount of the metal in ionic or salt form that is also present in the suspension solution. For example, colloidal silver may include a certain percentage of silver salt or ionic silver in solution, e.g., 10 wt % to 90 wt % of metal content can be ionic based on the total metal content. This being stated, certain metals for use in accordance with embodiments of the present disclosure are ruthenium, rhodium, osmium, iridium, palladium, platinum, copper, gold, silver, manganese, zinc, alloys thereof, and mixtures thereof. As mentioned, the transition metal can be colloidal or ionic. Colloidal metal can be elemental metal (or alloys of elemental metals), or can be in an insoluble salt form, such as zinc oxide or the like. For skin application, silver and zinc work well together, but metal choice can be dependent to some degree on the application, the levels of infection to be treated, etc.

It is also noted that any of these embodiments can often also benefit from the use of alloys. For example, certain combinations of metals in an alloy may provide benefits that are related more to other consideration, such as solution stability, substrate to be cleaned, etc. Examples of transition metal alloys for use in the present disclosure include but are not limited to copper-silver alloys, silver-manganese alloys, chromium-silver alloys, gold-silver alloys, magnesium-silver alloys, zinc-silver alloys, and the like.

Exemplary colloidal silvers that can be used in the first liquid composition include those sold by Solutions IE, Inc. under the trade names CS Plus and CS Ultra. Other colloidal silver products that can be used as the silver source include ASAP, Sovereign Silver, Silver Max, and the like. In one embodiment, the colloidal particles used in the present disclosure can have a particle size range of from 0.001 μm to 1.0 μm. In another embodiment, the colloidal transition metal particles can have a size range of from 0.030 μm to 0.5 μm. In still another embodiment the average particle size is 0.35 μm to 0.45 μm. If used in ionic form, silver salts can include but are not limited to silver nitrate, silver acetate, silver citrate, silver oxide, and/or silver carbonate. Though many colloidal silver solutions or ionic silver solutions that are functional for use in the formulations of the present disclosure can be used, in one embodiment, it can be desirable to use RO water as the suspension medium for the colloidal and/or ionic silver that is mixed with the other ingredients. In a more detailed aspect, the RO water can also be distilled, resulting in 18-20 MO water, though this is not required. Exemplary colloidal zinc that can be used includes that available from Purest Colloid, Inc. sold under the trade name MesoZinc, colloidal zinc available from Quality Colloids, Inc., and Zn1100 available from Solutions IE. Other sources of colloidal silver, colloidal zinc, and other colloidal or ionic metals are also generally available.

The peroxygen can be present in the compositions of the present disclosure at from 0.0001 wt % to 10 wt %, with the upper end of the range being modifiable as described previously herein. The peroxygen can be a single compound or a combination of multiple peroxygen compounds or peroxygen forming compounds. In one embodiment, the peroxygen can be any aliphatic or aromatic peracid (or peroxyacid) that is functional for treatment purposes in accordance with embodiments of the present disclosure. While any functional peroxyacid can be used, peroxyacids containing from 1 to 7 carbons are the most practical for use. These peroxyacids can include, but not be limited to, peroxyformic acid, peroxyacetic acid, peroxyoxalic acid, peroxypropanoic acid, perlactic acid, peroxybutanoic acid, peroxypentanoic acid, peroxyhexanoic acid, peroxyadipic acid, peroxycitric, and/or peroxybenzoic acid. The peroxyacid used in the present disclosure can be prepared using any method known in the art. When the peroxyacid is prepared from an acid and hydrogen peroxide, the resultant mixture contains both the peroxyacid and the corresponding acid that it is prepared from. For example, in embodiments that utilize peroxyacetic acid, the presence of the related acid (acetic acid) provides stability to the mixture, as the reaction is an equilibrium between the acid, hydrogen peroxide, and the peroxyacid and water, as follows:

H₂O₂+CH₃COOH [symbol] CH₃COO—OH+H₂O

Peracid salts, such as salts of the above listed peracids, can also be included as the peroxygen component of the solutions. Non-limiting examples of such salts include permanganates, perborates, perchlorates, peracetates, percarbonates, persulphates, and the like. The salts can be used alone or in combination with each other or other peroxygen compounds to form the peroxygen component of the disclosure.

In another embodiment, the peroxygen component of the disclosure can include a peroxide compound. While hydrogen peroxide is considered to be a desirable peroxide for use in accordance with embodiments of the present disclosure, other peroxides can also be used, such as metal peroxides and peroxyhydrates. The metal peroxides that can be used include, but are not limited to, sodium peroxide, magnesium peroxide, calcium peroxide, barium peroxide, and/or strontium peroxide. Other salts (for example sodium percarbonate) have hydrogen peroxide associated therewith much like waters of hydration, and these could also be considered to be a source of hydrogen peroxide, thereby producing hydrogen peroxide in situ. As mentioned above, the peroxides can be used alone or in combination with other peroxygen compounds to form the peroxygen component of the present disclosure.

Emollients, humectants, moisturizers, surfactants, skin nutrients, skin conditioners, skin protectants, and other skin health additives can also be used, such as carotenoids (e.g., astaxanthin), coconut oil as a moisturizer, and/or sorbitol or glycerol as an emollient as well as an alcohol. Likewise, avariety of humectants including those generally known in the art can be used. In one embodiment, the humectant can be a natural grain extract such as from wheat, oats, flax seed, and combinations thereof. Such natural extracts can be advantageous as they generally are less irritable to the skin. In one embodiment, the grain extract is gluten free. In another embodiment, the grain extract is a flax seed extract. Furthermore, examples of surfactants that can be used include, but are not limited to, liquid vegetable oil soap, sulfonated castor oil, sodium lauryl sulfate or any other liquid surfactant known in the art. Examples of skin protectants that can be used include, but are not limited to sunscreens, vitamin E, vitamin E derivatives, aloe vera gel, and combinations thereof.

The administration of the therapeutic aqueous composition can be done in any acceptable manner known in the medical and pharmaceutical arts. Specific non-limiting examples of topical administration include the use of fluids, aerosols, sprays, mists, lotions, creams, ointments, gels, gums, lozenges, suppository, drops, washes, dispensing bottles, squeeze tubes, pre-soaked fabric (cotton rounds, wipes, etc.), automatic mixing and/or dispensing devices, sprayers, bandages, transdermal patches or plasters, etc. Submersion of infected skin tissue is also an acceptable means of topical administration as well. The mode of administration can be dependent on the type and/or severity of the skin disease being treated and the formulated potency of the therapeutic aqueous composition. However, typically, it can be desirable to topically apply the aqueous composition to the areas where the infection is present or may shortly become present.

As stated above, the present disclosure is related to compositions, systems, and methods of treating (including prophylactically treating) skin disease. The amounts of the therapeutic aqueous compositions which can be administered using the methods of the present disclosure can vary depending on the type and location of the targeted infection, the mode of administration, and the potency or concentration of the aqueous composition administered. For example, when administered topically using a spray or submersion administration mode for topical local effect, the amount of aqueous composition may not be as important as the concentration of the aqueous composition and the frequency of administration may be more significant. In one embodiment, the administration can occur one or more times daily for a period of 1 day to 180 days. In another embodiment, the administration can occur one or more times daily for a period of 1 to 7 days. In another embodiment, the administration can occur one or more times for a period of 4 hours to 24 hours.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

### Compositions Usable for Treatment of Skin Disease or Infection

An aqueous composition is prepared which includes 0.1 wt % hydrogen peroxide, 4 wt % glycerol, 600 ppm of silver-zinc alloy, 7 wt % ethyl alcohol, 0.3 wt % salicylic acid, and the balance water. Optionally, additional skin health additives, such as emollients, carotenoids, skin nutrients, skin conditioners, skin protectants, and/or the like, can be substituted for a small portion of the water, e.g., up to 20 wt %. In one specific preparation scheme, the composition can be prepared by bringing two-parts (Part A and Part B) together prior to use and can be applied to the acne while reaction between Part A and Part B is occurring. For example, the silver-zinc alloy can be kept in Part A and the hydrogen peroxide can be kept in Part B. The other ingredients can typically be kept in either Part A and/or Part B.

### Example 2

### Compositions Usable for Treatment of Skin Disease or Infection

An aqueous composition is prepared which includes 0.05 wt % hydrogen peroxide acid, 8 wt % ethanol, 150 ppm by weight of colloidal silver, 0.5 wt % benzoyl peroxide, 3 wt % of a thickening agent (to form a non-runny gel or lotion), and the balance water. Optionally, additional skin health additives, such as emollients, carotenoids, skin nutrients, skin conditioners, skin protectants, and/or the like, can be substituted for a small portion of the water, e.g., up to 20 wt %. In one specific preparation scheme, the composition can be prepared by bringing two-parts (Part A and Part B) together prior to use and can be applied to the skin disease while reaction between Part A and Part B is occurring. For example, the silver-zinc alloy can be kept in Part A and the hydrogen peroxide can be kept in Part B. The other ingredients can typically be kept in either Part A and/or Part B.

### Example 3

### Compositions Usable for Treatment of Skin Disease or Infection

An aqueous composition is prepared which includes 10 wt % hydrogen peroxide, 2 wt % glycerol, 2 wt % sorbitol, 10,000 ppm of colloidal silver, 5,000 ppm colloidal zinc, 5 wt % ethyl alcohol, 1.5 wt % salicylic acid, and the balance water. Optionally, additional skin health additives, such as emollients, carotenoids, skin nutrients, skin conditioners, skin protectants, and/or the like, can be substituted for a small portion of the water, e.g., up to 20 wt %. In one specific preparation scheme, the composition can be prepared by bringing two-parts (Part A and Part B) together prior to use and can be applied to the skin disease while reaction between Part A and Part B is occurring. For example, the silver-zinc alloy can be kept in Part A and the hydrogen peroxide can be kept in Part B. The other ingredients can typically be kept in either Part A and/or Part B.

### Example 4

### Composition Usable for Treatment of Skin Disease or Infection

An aqueous composition suitable for treating skin disease is prepared which includes peroxide, alcohol, colloidal transition metal, a drug for treating skin disease, water, and other ingredients, as set forth in Table 1 below:

TABLE 1

| | | | QUANTITY ON | |
|---|---|---|---|---|
| | | | ROUND AFTER | |
| | | | SPRAYING PART | |
| | QUANTITY PER | | B ONTO | |
| | INDIVIDUAL | | PRE-SOAKED | |
| 2-PART FORMULA | PART | | ROUND | |
| Part A - Soaked On Cotton | 115 | ml | 69.7 | wt % |
| Rounds (30 count 2.25 inch | | | | |
| Cotton Round Swisspers ®) | | | | |
| Silver Colloids | 300 | ppm (wt) | 209.09 | ppm (wt) |
| Zinc Colloids | 100 | ppm (wt) | 69.70 | ppm (wt) |
| Glycerol | 5 | wt % | 3.48 | wt % |
| Denatured Ethyl Alcohol | 10 | wt % | 6.97 | wt % |
| Part B - Spray Activator | 50 | ml | 30.3 | wt % |
| Hydrogen Peroxide (dilute | 5 | wt % | 1.52 | wt % |
| from concentrate) | | | | |
| Coconut Oil | 4 | wt % | 1.21 | wt % |
| Salicylic Acid | 1 | wt % | 0.30 | wt % |
| Skin Health Additives¹ | 0.5 | wt % | 0.15 | wt % |

¹Exemplary Skin Health Addtives that can be used include benzoic peroxide, carotenoids (e.g., astaxanthin), skin nutrients, skin conditioners, skin protectants, and/or emollients.
Water is present in Part A and Part B up to 100 wt %.

### Example 5

### Composition Usable for Treatment of Skin Disease or Infection

An aqueous composition suitable for treating skin disease is prepared which includes peroxide, alcohol, colloidal transition metal, a drug for treating skin disease, water, and other ingredients, as set forth in Table 1 below:

TABLE 2

| | | | QUANTITY ON | |
|---|---|---|---|---|
| | | | ROUND AFTER | |
| | | | SPRAYING PART | |
| | QUANTITY PER | | B ONTO | |
| | INDIVIDUAL | | PRE-SOAKED | |
| 2-PART FORMULA | PART | | ROUND | |
| Part A - Soaked On Cotton | 115 | ml | 69.7 | wt % |
| Rounds (30 count 2.25 inch | | | | |
| Cotton Round Swisspers ®) | | | | |
| Silver Colloids | 400 | ppm (wt) | 278.79 | ppm (wt) |
| Glycerol | 5 | wt % | 3.48 | wt % |
| Denatured Ethyl Alcohol | 10 | wt % | 6.97 | wt % |
| Part B - Spray Activator | 50 | ml | 30.3 | wt % |
| Hydrogen Peroxide (dilute | 3 | wt % | 0.91 | wt % |
| from concentrate) | | | | |
| Coconut Oil | 4 | wt % | 1.21 | wt % |
| Benzoyl Peroxide | 3 | wt % | 0.90 | wt % |
| Skin Health Additives¹ | 0.5 | wt % | 0.15 | wt % |

¹Exemplary Skin Health Addtives that can be used include benzoic peroxide, carotenoids (e.g., astaxanthin), skin nutrients, skin conditioners, skin protectants, and/or emollients.
Water is present in Part A and Part B up to 100 wt %.

### Example 6

### Composition Usable for Treatment of Skin Disease or Infection

An aqueous composition suitable for treating skin disease is prepared which includes peroxide, alcohol, colloidal transition metal, a drug for treating skin disease, water, and other ingredients, as set forth in Table 1 below:

TABLE 3

| | | | QUANTITY ON | |
|---|---|---|---|---|
| | | | ROUND AFTER | |
| | | | SPRAYING PART | |
| | QUANTITY PER | | B ONTO | |
| | INDIVIDUAL | | PRE-SOAKED | |
| 2-PART FORMULA | PART | | ROUND | |
| Part A - Soaked On Cotton | 85 | mL | 61.3 | wt % |
| Rounds (30 count 2.25 inch | | | | |
| Cotton Round Swisspers ®) | | | | |
| Salicylic Acid | 2 | wt % | 1.23 | wt % |
| Glycerol | 3.5 | wt % | 2.15 | wt % |
| Hydrogen Peroxide (dilute | 5 | wt % | 3.06 | wt % |
| from concentrate) | | | | |
| Ethyl Alcohol | 11 | wt % | 6.74 | wt % |
| Coconut Oil | 6 | wt % | 3.68 | wt % |
| Skin Health Additives¹ | 0.1 | wt % | 0.06 | wt % |
| Part B - Spray Activator | 60 | mL | 38.7 | wt % |
| Silver Colloids (449 ppm by | 80 | wt % (metal | 139.05 | wt % |
| weight) | | content) | | |
| Zinc Colloids (1100 ppm by | 20 | wt % (metal | 85.16 | wt % |
| weight) | | content) | | |

¹Exemplary Skin Health Addtives that can be used include benzoic peroxide, carotenoids (e.g., astaxanthin), skin nutrients, skin conditioners, skin protectants, and/or emollients.
Water is present in Part A and Part B up to 100 wt %.

### Example 7

### Composition Usable for Treatment of Skin Disease or Infection

An aqueous composition suitable for treating skin disease is prepared which includes peroxide, alcohol, colloidal transition metal, a drug for treating skin disease, water, and other ingredients, as set forth in Table 1 below:

TABLE 4

| | | | QUANTITY ON | |
|---|---|---|---|---|
| | | | ROUND AFTER | |
| | | | SPRAYING PART | |
| | QUANTITY PER | | B ONTO | |
| | INDIVIDUAL | | PRE-SOAKED | |
| 2-PART FORMULA | PART | | ROUND | |
| Part A - Soaked On Cotton | 85 | mL | 61.3 | wt % |
| Rounds (30 count 2.25 inch | | | | |
| Cotton Round Swisspers ®) | | | | |
| Glycerol | 5.5 | wt % | 3.38 | wt % |
| Hydrogen Peroxide (dilute | 5 | wt % | 3.06 | wt % |
| from concentrate) | | | | |
| Ethyl Alcohol | 11 | wt % | 6.74 | wt % |
| Coconut Oil | 6 | wt % | 3.68 | wt % |
| Skin Health Additives¹ | 0.1 | wt % | 0.06 | wt % |
| Part B - Spray Activator | 60 | mL | 38.7 | wt % |
| Silver Colloids (449 ppm by | 80 | wt % (metal | 139.05 | wt % |
| weight) | | content) | | |
| Zinc Colloids (1100 ppm by | 20 | wt % (metal | 85.16 | wt % |
| weight) | | content) | | |

¹Exemplary Skin Health Addtives that can be used include benzoic peroxide, carotenoids (e.g., astaxanthin), skin nutrients, skin conditioners, skin protectants, and/or emollients.
Water is present in Part A and Part B up to 100 wt %.

### Example 8

### Treatment of Acne

Three separate studies were conducted using a formulation similar to that described in Examples 1-7, as set forth below:
a) A subject used a two-part composition daily by admixing the two-parts and immediately applying the resultant composition daily to the skin for a period of 2 weeks. The subject experienced a 95% reduction in my acne.
b) A subject that suffered from acne for over 10 years that had tried many products to limited success applied a two-part composition (immediately after admixing) to the face twice per day for 2 weeks. Most of the acne was gone at the end of the 2 week period.
c) A subject used a two-part composition daily by admixing the two-parts and immediately applying the resultant composition daily to the skin for a period of 1 month. The subject has been clear since the treatment ended.

### Example 9

### Treatment of Warts

Two separate studies were conducted using a formulation similar to that described in Examples 1-7, as set forth below:
a) A subject had a wart on the side of the cheek for over 3 years. After admixing and applying a two-part composition to the wart a few times a day for 4 days, the wart fell off.
b) A subject with many warts on the back began applying a two-part product (after admixing) to the warts daily. Within 2 weeks, the warts were nearly gone.

### Example 10

### Treatment of Fungal Infections

A study was conducted using a formulation similar to that described in Examples 1-7, as follows. A subject had an ugly fungal infection (dry, scaly patches) in many locations on the face. Within 3 days of applying the product (two-part composition admixed prior to use), the fungal patches were mostly clear. The subject continued using the product over a few weeks and the fungus ultimately cleared completely.

### Example 11

### Treatment of Bacterial Infection

A study was conducted using a formulation similar to that described in Examples 1-7, as follows. A subject reported that as child, the skin on the hands started to crack and peel. Doctors prescribed the use of plastic gloves with Nieva cream, which did not work, and often, became worse. The subject is lactose and glucose intolerant which causes leaky gut syndrome. After beginning to use the product for a period of 1 week, the subject's hands looked and felt significantly better.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A composition suitable for treating skin disease, comprising:
water,
from 0.0001 wt % to 10.0 wt % of a peroxygen;
from 0.0001 ppm to 50,000 ppm by weight of a transition metal or alloy thereof; and
a drug suitable for treating the skin disease.

2. The composition of claim 1, wherein the drug includes a member selected from the group consisting of benzoyl peroxide, salicylic acid, sulfur, resorcinol, resorcinol monoacetate, amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, undecylenate, acyclovir, penciclovir, famciclovir, valacyclovir, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, rimantadine, zanamivir, erythromycin, clindamycin, tetracycline, bacitracin, neomycin, mupirocin, polymyxin B, quinolones, and imiquimod.

3. The composition of claim 1, wherein the drug includes benzoyl peroxide.

4. The composition of claim 1, wherein the drug includes salicylic acid.

5. The composition of claim 1, wherein the drug includes sulfur.

6. The composition of claim 1, wherein the drug includes resorcinol or resorcinol monoacetate.

7. The composition of claim 6, wherein the drug further includes sulfur.

8. The composition of claim 1, wherein the transition metal or alloy thereof is selected from the group consisting of ruthenium, rhodium, osmium, iridium, palladium, platinum, copper, gold, silver, manganese, zinc, alloys thereof, and mixtures thereof.

9. The composition of claim 1, wherein the transition metal or alloy thereof is a colloidal transition metal or alloy thereof.

10. The composition of claim 9, wherein the colloidal transition metal or alloy thereof includes colloidal silver.

11. The composition of claim 9, wherein the colloidal transition metal or alloy thereof includes colloidal zinc.

12. The composition of claim 9, wherein the colloidal transition metal or alloy thereof includes a mixture or alloy of colloidal zinc and colloidal silver.

13. The composition of claim 9, wherein the colloidal transition metal or alloy thereof has an average particle size of from 0.030 μm to 0.5 μm.

14. The composition of claim 1, wherein the transition metal or alloy thereof is an ionic transition metal.

15. The composition of claim 1, wherein the transition metal or alloy thereof is present at from 0.0001 ppm to 1,500 ppm by weight.

16. The composition of claim 1, wherein the peroxygen is a peracid.

17. The composition of claim 16, wherein the peracid is selected from the group consisting of peroxyformic acid, peroxyacetic acid, peroxyoxalic acid, peroxypropanoic acid, perlactic acid, peroxybutanoic acid, peroxypentanoic acid, peroxyhexanoic acid, peroxyadipic acid, peroxycitric, peroxybenzoic acid, and mixtures thereof.

18. The composition of claim 1, wherein the peroxygen is a peroxide.

19. The composition of claim 1, wherein the peroxygen includes a peracid and a peroxide.

20. The composition of claim 1, wherein the peroxygen is present at from 0.05 wt % to 5.0 wt %.

21. The composition of claim 1, wherein the peroxygen is present at from 0.1 wt % to 1.5 wt %.

22. The composition of claim 1, further comprising an alcohol.

23. The composition of claim 22, wherein the alcohol includes a member selected from the group consisting of methanol, ethanol, a propanol, a butanol, a pentanol, and mixtures thereof.

24. A method as in claim 22, wherein the alcohol includes a polyhydric alcohol.

25. The composition of claim 22, wherein the alcohol includes glycerol.

26. The composition of claim 1, formulated as an ointment, cream, mouth rinse, gel, lozenge, gum, wipe, dermal patch, foam, powder, aerosol, or bandage dressings.

27. The composition of claim 1, further comprising at least one skin health additive selected from the group consisting of emollients, carotenoids, skin nutrients, skin conditioners, and skin protectants.

28. The composition of claim 1, in the form of a reacting formulation, wherein at least two components of the composition are not in equilibrium at the time of application.

29. The composition of claim 1, comprising from 0.1 wt % to 10 wt % salicylic acid as the drug, from 0.1 wt % to 10 wt % hydrogen peroxide as the peroxygen, from 1 ppm to 20,000 ppm by weight colloidal silver as a first portion of the transition metal, from 1 ppm to 20,000 ppm by weight colloidal zinc as a second portion of the transition metal, and wherein the composition further comprises from 0.5 wt % to 25 wt % glycerol and from 0.5 wt % to 15 wt % coconut oil.

30. A method of treating a skin disease, comprising applying the composition of claim 1 directly to a skin site afflicted with the skin disease.

31. A system suitable for treating skin disease, comprising:
a first container containing Part A of a two-part solution, Part A including a transition metal or alloy thereof;
a second container containing Part B of the two-part solution, Part B including water and a peroxygen; and
a drug suitable for treating the skin disease present in at least one of Part A, Part B, or a third formulation,
wherein upon combining Part A and Part B, a reacting formulation is formed that, in combination with the drug, is effective for treating the skin disease.

32. The system of claim 31, wherein the drug includes a member selected from the group consisting of benzoyl peroxide, salicylic acid, sulfur, resorcinol, resorcinol monoacetate, amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, undecylenate, acyclovir, penciclovir, famciclovir, valacyclovir, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, rimantadine, zanamivir, erythromycin, clindamycin, tetracycline, bacitracin, neomycin, mupirocin, polymyxin B, quinolones, and imiquimod.

33. The system of claim 31, wherein the drug includes benzoyl peroxide.

34. The system of claim 31, wherein the drug includes salicylic acid.

35. The system of claim 31, wherein the drug includes sulfur.

36. The system of claim 31, wherein the drug includes resorcinol or resorcinol monoacetate.

37. The system of claim 36, wherein the drug further includes sulfur.

38. The system of claim 31, wherein the transition metal or alloy thereof is selected from the group consisting of ruthenium, rhodium, osmium, iridium, palladium, platinum, copper, gold, silver, manganese, zinc, alloys thereof, and mixtures thereof.

39. The system of claim 31, wherein the transition metal or alloy thereof is a colloidal transition metal or alloy thereof.

40. The system of claim 39, wherein the colloidal transition metal or alloy thereof includes colloidal silver.

41. The system of claim 39, wherein the colloidal transition metal or alloy thereof includes colloidal zinc.

42. The system of claim 39, wherein the colloidal transition metal or alloy thereof includes a mixture or alloy of colloidal zinc and colloidal silver.

43. The system of claim 39, wherein the colloidal transition metal or alloy thereof has an average particle size of from 0.030 μm to 0.5 μm.

44. The system of claim 31, wherein the transition metal or alloy thereof is an ionic transition metal.

45. The system of claim 31, wherein the transition metal or alloy thereof is present in the reacting formulation at from 0.0001 ppm to 50,000 ppm by weight.

46. The system of claim 31, wherein the transition metal or alloy thereof is present in the reacting formulation at from 0.0001 ppm to 1,500 ppm by weight.

47. The system of claim 31, wherein the peroxygen is a peracid.

48. The system of claim 47, wherein the peracid is selected from the group consisting of peroxyformic acid, peroxyacetic acid, peroxyoxalic acid, peroxypropanoic acid, perlactic acid, peroxybutanoic acid, peroxypentanoic acid, peroxyhexanoic acid, peroxyadipic acid, peroxycitric, peroxybenzoic acid, and mixtures thereof.

49. The system of claim 31, wherein the peroxygen is a peroxide.

50. The system of claim 31, wherein the peroxygen includes a peracid and a peroxide.

51. The system of claim 31, wherein the peroxygen is present in the reacting formulation at from 0.0001 wt % to 10.0 wt % of a peroxygen.

52. The system of claim 31, wherein the peroxygen is present in the reacting formulation at from 0.05 wt % to 5.0 wt %.

53. The system of claim 31, wherein the peroxygen is present in the reacting formulation at from 0.1 wt % to 1.5 wt %.

54. The system of claim 31, further comprising an alcohol.

55. The system of claim 54, wherein the alcohol includes a member selected from the group consisting of methanol, ethanol, propanols, butanols, pentanols, and mixtures thereof.

56. A method as in claim 54, wherein the alcohol includes a polyhydric alcohol.

57. The system of claim 54, wherein the alcohol includes glycerol.

58. The system of claim 31, further comprising at least one skin health additive present in Part A or Part B.

59. The system of claim 58, wherein the skin health additive is present in Part B and is selected from the group consisting of emollients, carotenoids, skin nutrients, skin conditioners, and skin protectants.

60. The system of claim 31, wherein the first container contains a plurality of skin wipe pre-soaked with Part A, and the second container is a dispenser adapted to dispense Part B onto a skin wipe to form a reacting formulation that is effective for treating the skin disease.

61. The system of claim 31, wherein the drug is present in Part A.

62. The system of claim 31, wherein the drug is present in Part B.

63. The system of claim 31, wherein the drug is present in the third formulation and is applied to the skin along with the reacting formulation.

64. A method of treating a skin disease, comprising:
obtaining the system of claim 31;
combining Part A and Part B in the presence of the drug to form a reacting formulation; and
applying the reacting formulation to a skin site afflicted with the skin disease.

65. A system suitable for treating skin disease, comprising:
a first container containing Part A of a two-part solution, Part A including a transition metal or alloy thereof; and
a second container containing Part B of the two-part solution, Part B including water and a peroxygen,
wherein one of Part A and Part B is soaked into a skin wipe, and the other of Part A and Part B is present in a dispenser adapted to dispense its solution onto the skin wipe to form a reacting formulation that is effective for treating the skin disease.

66. The system of claim 65, wherein further comprising a drug present in Part B, the drug including a member selected from the group consisting of benzoyl peroxide, salicylic acid, sulfur, resorcinol, resorcinol monoacetate, amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, undecylenate, acyclovir, penciclovir, famciclovir, valacyclovir, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, rimantadine, zanamivir, erythromycin, clindamycin, tetracycline, bacitracin, neomycin, mupirocin, polymyxin B, quinolones, and imiqui mod.

67. The system of claim 65, wherein the transition metal or alloy thereof is selected from the group consisting of ruthenium, rhodium, osmium, iridium, palladium, platinum, copper, gold, silver, manganese, zinc, alloys thereof, and mixtures thereof.

68. The system of claim 65, wherein the transition metal or alloy thereof is a colloidal transition metal or alloy thereof.

69. The system of claim 68, wherein the colloidal transition metal or alloy thereof includes colloidal silver.

70. The system of claim 68, wherein the colloidal transition metal or alloy thereof includes colloidal zinc.

71. The system of claim 68, wherein the colloidal transition metal or alloy thereof includes a mixture or alloy of colloidal zinc and colloidal silver.

72. The system of claim 68, wherein the colloidal transition metal or alloy thereof has an average particle size of from 0.030 μm to 0.5 μm.

73. The system of claim 65, wherein the transition metal or alloy thereof is an ionic transition metal.

74. The system of claim 65, wherein the transition metal or alloy thereof is present in the reacting formulation at from 0.0001 ppm to 50,000 ppm by weight.

75. The system of claim 65, wherein the transition metal or alloy thereof is present in the reacting formulation at from 0.0001 ppm to 1,500 ppm by weight.

76. The system of claim 65, wherein the peroxygen is a peracid.

77. The system of claim 76, wherein the peracid is selected from the group consisting of peroxyformic acid, peroxyacetic acid, peroxyoxalic acid, peroxypropanoic acid, perlactic acid, peroxybutanoic acid, peroxypentanoic acid, peroxyhexanoic acid, peroxyadipic acid, peroxycitric, peroxybenzoic acid, and mixtures thereof.

78. The system of claim 65, wherein the peroxygen is a peroxide.

79. The system of claim 65, wherein the peroxygen includes a peracid and a peroxide.

80. The system of claim 65, wherein the peroxygen is present in the reacting formulation at from 0.0001 wt % to 10.0 wt % of a peroxygen.

81. The system of claim 65, wherein the peroxygen is present in the reacting formulation at from 0.05 wt % to 5.0 wt %.

82. The system of claim 65, wherein the peroxygen is present in the reacting formulation at from 0.1 wt % to 1.5 wt %.

83. The system of claim 65, further comprising an alcohol.

84. The system of claim 83, wherein the alcohol includes a member selected from the group consisting of methanol, ethanol, propanols, butanols, pentanols, and mixtures thereof.

85. A method as in claim 83, wherein the alcohol includes a polyhydric alcohol.

86. The system of claim 83, wherein the alcohol includes glycerol.

87. The system of claim 65, further comprising at least one skin health additive present in Part A or Part B.

88. The system of claim 87, wherein the skin health additive is present in Part B and is selected from the group consisting of emollients, carotenoids, skin nutrients, skin conditioners, and skin protectants.

89. The system of claim 87, wherein Part B includes coconut oil.

90. The system of claim 85, wherein one of Part A and Part B is soaked into a plurality of skin wipes, and the other of Part A and Part B is present in the dispenser adapted to dispense its solution onto less than all of the plurality of skin wipes so that not all of the plurality of skin wipes are activated.

91. The system of claim 85, wherein less than all of the plurality of skin wipes is a single skin wipe that is activated while the remaining skin wipes remain unactivated.

92. A method of treating a skin disease, comprising:
obtaining a two-part solution comprising Part A which includes a transition metal or alloy thereof, and Part B which includes water and a peroxygen;
combining Part A with Part B to form a reacting formulation; and
applying the reacting formulation to the skin disease.

93. The method of claim 92, wherein the skin disease is an inflammatory infection.

94. The method of claim 93, wherein the inflammatory disease includes acne, aczema, dermatitis, poison ivy, psoriasis, pyoderma gangrenosum, rosacea, hives, or burns.

95. The method of claim 92, wherein the skin disease is a bacterial skin infection.

96. The method of claim 95, wherein the bacterial skin infection includes impetigo, folliculitis, furunculosis, carbunculosis, eethyma, erysipelas, cellulitis, or necrotizing fasciitis.

97. The method of claim 92, wherein the skin disease includes a fungal or yeast infection.

98. The method of claim 97, wherein the fungal or yeast infection includes dermatophytosis, candidiasis, tinea, athlete's foot, nail fungal infection, or diaper rash.

99. The method of claim 92, wherein the skin disease includes a viral infection.

100. The method of claim 99, wherein the viral infection includes herpes simplex, herpes zoster, cold sores, warts, or molluscum contagiosum.

101. The method of claim 92, wherein the skin disease includes an infection caused by small macro organism selected from mites, insects, and bugs.

102. The method of claim 92, wherein the transition metal or alloy thereof is a colloidal transition metal.

103. The method of claim 92, wherein Part A further comprises an alcohol.

104. The method of claim 92, wherein the peroxygen is a peroxide.

105. The method of claim 92, further comprising a drug suitable for treating the skin disease present in at least one of Part A, Part B, or a third formulation, wherein upon combining Part A and Part B, a reacting formulation is formed that, in combination with the drug, is effective for treating the skin disease.

106. The method of claim 105, wherein the drug is includes a member selected from the group consisting of benzoyl peroxide, salicylic acid, sulfur, resorcinol, resorcinol monoacetate, amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, undecylenate, acyclovir, penciclovir, famciclovir, valacyclovir, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, rimantadine, zanamivir, erythromycin, clindamycin, tetracycline, bacitracin, neomycin, mupirocin, polymyxin B, quinolones, and imiquimod.

107. The method of claim 105, wherein the drug is includes a member selected from the group consisting of benzoyl peroxide, salicylic acid, sulfur, resorcinol, resorcinol monoacetate, and combinations thereof.
